(19) United States
(12) Patent Application Publication
(10) Pub. No.: US 2010/0137907 A1
(43) Pub. Date: Jun. 3, 2010

(54) Power Operated Chiropractic Adjuster

(76) Inventor: Stephen Tsai, Caotun Town (TW)

Correspondence Address: Wang Law Firm, Inc., 4989 Peachtree Parkway,, Suite 200, Norcross, GA, 30092, US

(21) Appl. No.: 12/327,151

(22) Filed: Dec. 3, 2008

(51) Int. Cl. A61F 5/00

(52) U.S. Cl. 606/238

(57) ABSTRACT

A power operated chiropractic adjuster is disclosed. It uses a driving set to rotate an urging ring, which drives a pulling ring to perform a reciprocal motion along its axial direction. It has an impact set that includes a control pin, a plunger, and an escape tube connected with the pulling ring to perform a reciprocal motion with the pulling ring. The rear section of the firing ring has a second spring. The escape tube is mounted on the plunger and constrains it. The control pin is disposed inside the plunger and can release the constraint between the plunger and the escape tube. The plunger is driven by a restoring elastic force of the second spring to perform a firing action. The power operated chiropractic adjuster also has an urging pin set whose one end is disposed opposite to the impact set and whose other end is opposite to a hammer set. When the plunger fires, the urging pin set is hit by the plunger to push the hammer set, producing an ejection effect.

### BACKGROUND OF THE INVENTION

1. Field of Invention

The invention relates to a massage device and, in particular, to a power operated chiropractic adjuster.

2. Related Art

As shown in FIG. 12, U.S. Pat. No. 7,144,417 discloses a power operated massage gun structure. It utilizes an electromagnetic driving method to drive a plunger 1 to hit a hammer set 2. This produces an impact on the human spine to achieve the massage effect. However, it has the following disadvantages:

1. The power operated massage gun structure makes use of electromagnetic means to drive the plunger 1. Its instantaneous impact is very large. If this force is directly imposed on a weaker part of the human spine, it may result in a serious or permanent injury.

2. The power operated massage gun structure therein uses an external power cord 3 to provide the power required by the electromagnetic means. It therefore requires a corresponding power outlet for normal uses. In other words, if there is no suitable power outlet, it cannot function normally. Furthermore, the power cord 3 extended from the power operated massage gun is inconvenient in usual operations.

### SUMMARY OF THE INVENTION

An objective of the invention is to provide a power operated chiropractic adjuster that has a softer instantaneous impact and exerts a smoother force on the human spine for massaging. This effectively prevents the spine from injuries due to large forces.

Another objective of the invention is to provide a power operated chiropractic adjuster that can adjust the magnitude of exerting force.

A further objective of the invention is to provide a power operated chiropractic adjuster that uses an independent power source so that it is not limited by the environmental conditions.

To achieve the above-mentioned objectives, the disclosed power operated chiropractic adjuster includes: a body, a urging ring, a pulling ring, an impact set, and an urging pin set.

The body has a driving set and a power supply inside. The power supply is electrically connected with the driving set.

One end of the urging ring is connected with the driving set and driven by the driving set to perform a rotational motion. The other end of the urging ring has an accommodating space containing two oppositely disposed actuating parts. The accommodating space is formed with an urging surface.

The pulling ring is disposed in the accommodating space of the urging ring. A first spring is interposed between the pulling ring and the urging surface of the accommodating space. A connecting hole is formed along the axial direction of the pulling ring. The rim of the pulling ring is provided with two actuating blocks, each of which has an urging slant surface. The two actuating parts on the urging ring and the urging slant surfaces of the two actuating blocks are engaged. When the urging ring is rotated by the driving set, the actuating parts on the urging ring urge against the urging slant surfaces on the pulling ring, forcing the pulling to move along its axial direction toward the urging surface of the urging ring. The first spring is gradually compressed. When the actuating parts of the urging ring leave the urging slant surfaces of the pulling ring, the pulling ring is pushed by the resilient force of the first spring back to its original position.

The impact set is disposed in the connecting hole of the pulling ring. It mainly includes a control pin, a hollow plunger, and an escape tube mounted in sequence. The plunger has a front section and a rear section. The plunger is formed with at least one constraining hole. A second spring is interposed between the rear section of the plunger and the urging surface of the urging ring. The control pin goes through the inside of the plunger. The control pin has a constraining section and an escaping section. The escape tube is mounted on the front section of the plunger. The escape tube is connected with the pulling ring to perform a reciprocal motion with it. A steel ball is disposed inside the plunger and corresponds to the constraining hole. When the steel ball and the constraining section of the control pin are in contact, the steel ball urges against the rim of the escape tube through the constraining hole of the plunger. The plunger and the escape tube thus constrain each other. When the escape tube moves with the pulling ring toward the urging surface of the accommodating space of the urging ring, it concurrently pulls the plunger to move toward the urging surface and compresses the second spring. When the steel ball is in contact with the escaping section of the control pin, it urges against the rim of the escape tube without through the constraining hole. In this case, the plunger is driven by the resilient force of the second spring to perform a firing action.

One end of the urging pin set is opposite to the impact set. Its other end is opposite to a hammer set. When the plunger of the impact set produces a firing action, the urging pin set is hit by the plunger to push the hammer set. This produces an ejecting force to massage the human body.

Moreover, the urging pin set consists of an urging pin tube and an urging pin. The urging pin tube has a central hole extending along its axial direction. The urging pin is disposed inside the central hole. One end of the urging pin has a pin head, and the other end has a surging ring. The pin head is opposite to the hammer set, and the surging ring is opposite to the impact set. An urging pin spring is interposed between the surging ring and the urging pin tube to urge against the surging ring and move the urging pin toward the impact set. An adjusting bar is disposed from the urging pin head toward the urging pin tube. The central hole of the urging pin tube is surrounded by multiple adjusting holes, the depths of which are all different. An adjusting tube is further mounted outside the urging pin head. By rotating the adjusting tube, the urging pin head is brought opposite to one of the adjusting holes.

### BRIEF DESCRIPTION OF THE DRAWINGS

The invention will become more fully understood from the detailed description given herein below illustration only, and thus is not limitative of the present invention, and wherein:

FIG. 1 is a three-dimensional exploded view of the invention;

FIG. 2 is a schematic structural view after the invention is assembled;

FIG. 3 is a schematic view of the invention in use, showing that the driving set starts to act when the user urges the hammer set against his body;

FIG. 4 is a schematic view of the invention in use, showing that the actuating parts of the urging ring push the urging slant surfaces of the pulling ring to force it to move;

FIG. 5 is a schematic view of the invention in use, showing that the control pin moves toward and urges against the urging surface of the urging ring;

FIG. 6 is a schematic view of the invention in use, showing that the steel ball moves from the constraining section of the control pin to the escaping section;

FIG. 7 is a schematic view of the invention in use, showing that the plunger is driven by the resilient force of the second spring to perform a firing action;

FIG. 8 is a schematic view of the invention in use, showing that when the user rotates the adjusting tube so that the adjust bar of the urging pin head corresponds to an adjusting hole of a larger depth;

FIG. 9 is a schematic view of the invention in use, showing that the adjusting bar of the urging pin head is inserted into an adjusting hole with a larger depth;

FIG. 10 is a three-dimensional exploded view of the second embodiment of the invention;

FIG. 11 is a local cross-sectional view of the second embodiment, showing the composition of the hammer set; and

FIG. 12 is a schematic view of the power operated massage gun disclosed in U.S. Pat. No. 7,144,417.

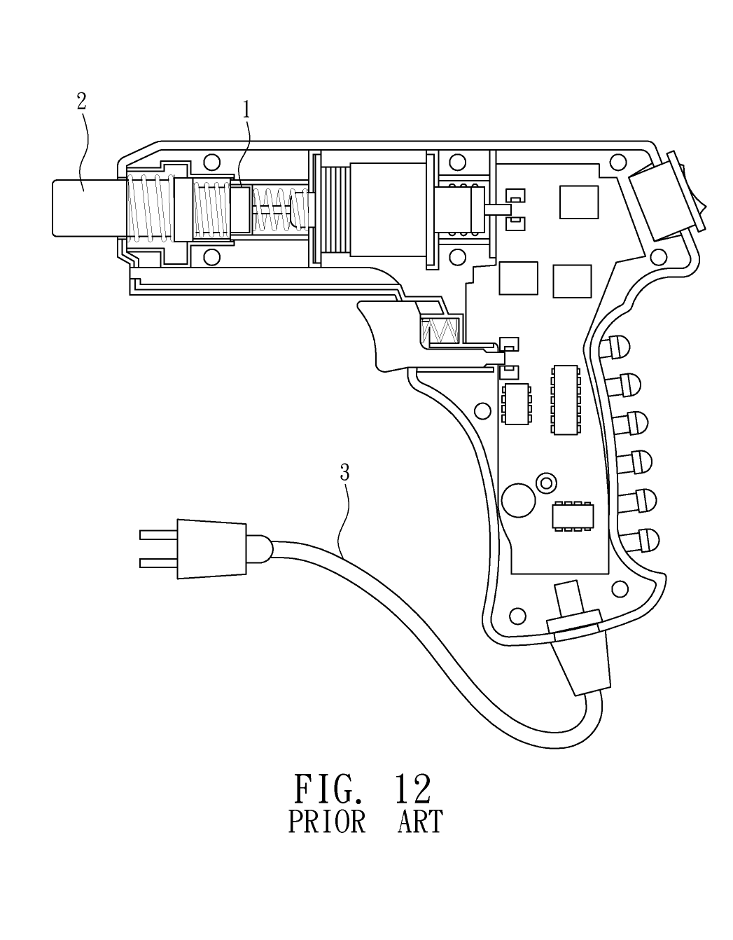

### DETAILED DESCRIPTION OF THE INVENTION

The present invention will be apparent from the following detailed description, which proceeds with reference to the accompanying drawings, wherein the same references relate to the same elements.

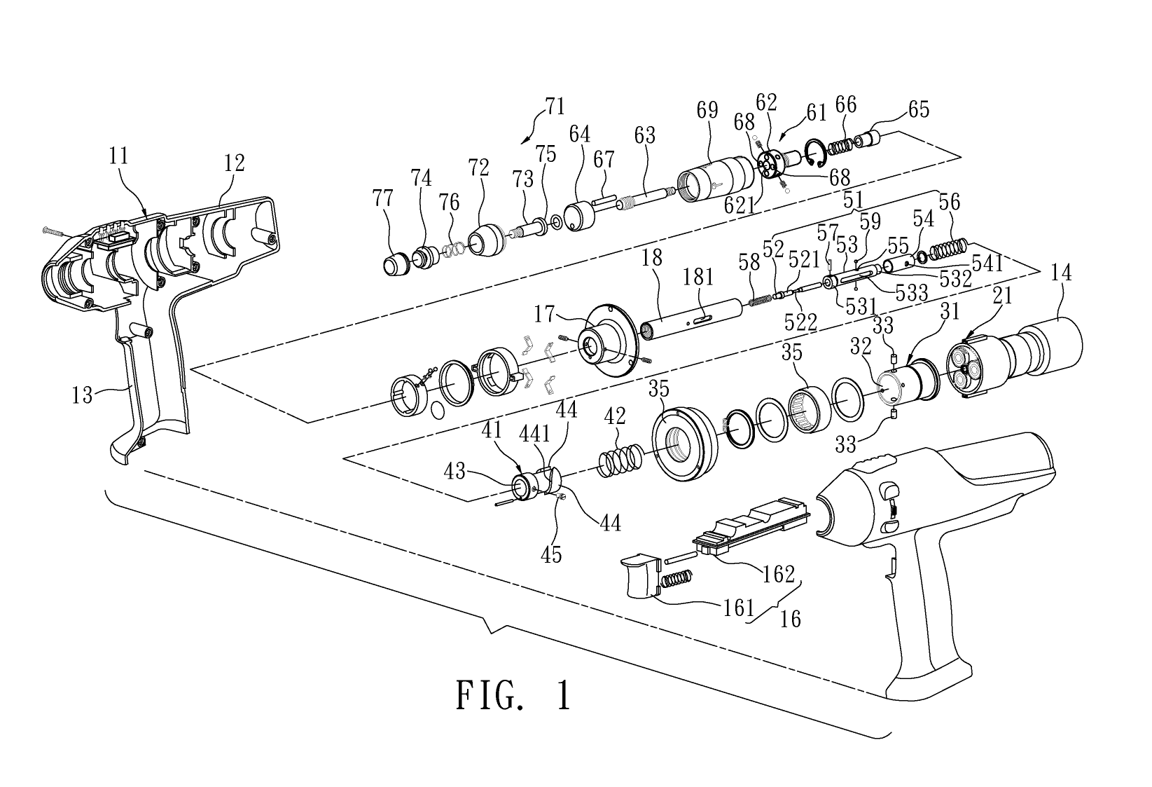

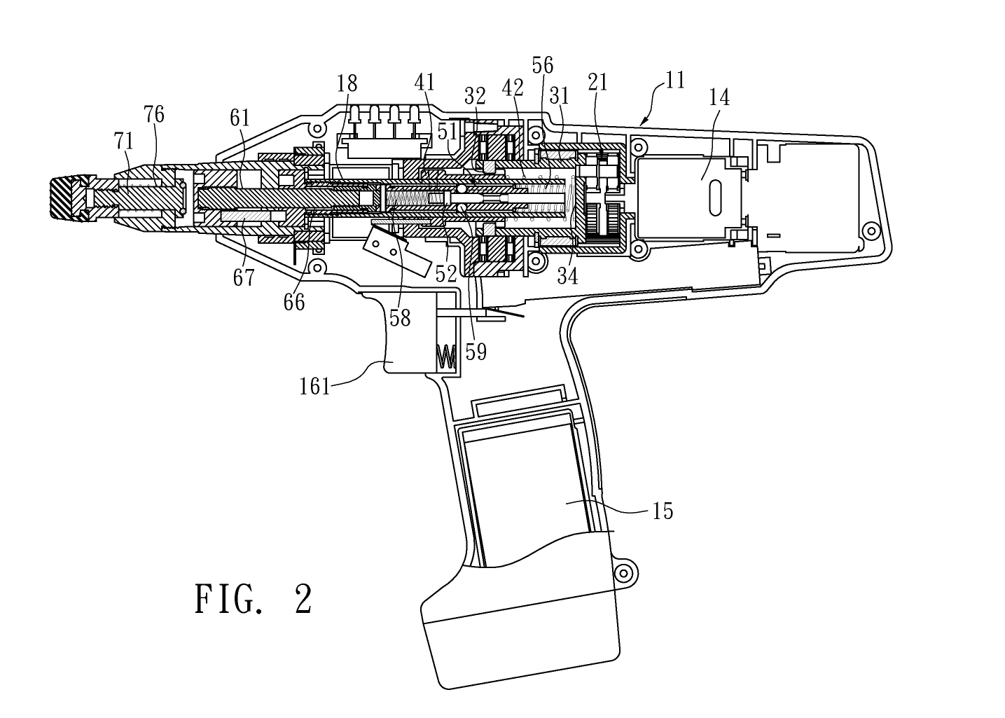

Please refer to FIGS. 1 and 2 for a first embodiment of the invention. This embodiment serves only for the illustration purpose. The invention is not restricted to this particular structure.

The disclosed power operated chiropractic adjuster comprises: a body 11, a gear set 21, a urging 31, a pulling ring 41, an impact set 51, a urging pin set 61, and a hammer set 71.

The body 11 has a pistol shape with a gun body 12 and a gun grip 13. A driving set 14 consisting of a braking motor is disposed inside the gun body 12. A power supply 15 consisting of batteries is disposed inside the gun grip 13. The gun body 12 of the body 11 further has an ON/OFF switch 16 inside. The ON/OFF switch 16 is electrically connected with the driving set 14 and the power supply 15 to control the electricity conduction in between. When the driving set 14 and the power supply 15 are in electrical communication, the driving set 14 starts to act. In this embodiment, the ON/OFF switch 16 includes a depressing part 161 and a micro switch 162 activated by the depressing part 161. One side of the depressing part 161 exposes from the gun grip 13 of the body 11. The micro switch 162 is disposed inside the gun body 12 of the body 11.

The gear set 21 consists of several decelerating gears. The driving set 14 is connected with the gear set 21 to drive it.

The urging ring 31 is connected with the gear set 21 by its one end. When the driving set 14 acts, it first drives the gear set 21 to operate. Afterwards, the gear set 21 rotates the urging ring 31. The other end of the urging ring 31 has an accommodating space 32. The accommodating space 32 has two protruding and oppositely disposed actuating parts 33 inside. An urging surface 34 is formed on one end of the accommodating space 32. Several bearings 35 are interposed between the outer rim of the urging ring 31 and the inside of the body 11.

The pulling ring 41 is disposed in the accommodating space 32 of the urging ring 31. A first spring 42 is interposed between the pulling ring 41 and the urging surface 34 of the accommodating space 32. A connecting hole 43 is formed along the axial direction of the pulling ring 41. Along the rim of the pulling ring 41 has two actuating blocks 44, each of which has an urging slant surface 441. The two actuating parts 33 on the urging ring 31 engage with the urging slant surfaces 441 of the two actuating blocks 44. When the driving set 14 drives the gear set 21 to rotate the urging ring 31, the actuating parts on the urging ring 31 urge against the urging slant surfaces 441 on the pulling ring 41. The pulling ring 41 is forced to move along its axial direction toward the urging surface 34 of the urging ring 31. The first spring 42 is gradually compressed. When the actuating parts 33 of the urging ring 31 leave the urging slant surfaces 441 of the pulling ring 41, the pulling ring 41 is restored by the resilient force of the first spring 42 to its original position.

The impact set 51 is disposed in the connecting hole 43 of the pulling ring 41. The impact set 51 mainly comprises a control pin 52, a hollow plunger 53, and an escape tube 54 mounted in sequence. The plunger 53 has a front section 531 and a rear section 532 and at least one constraining hole 55. A second spring 56 is interposed between the rear section 532 of the plunger 53 and the urging surface 34 of the urging ring 31. The control pin 52 is disposed inside the plunger 53. A pin 57 is disposed in the front section 531 of the plunger 53. A third spring is interposed between the pin 57 and the control pin 52. The control pin 52 has a constraining section 521 and an escaping section 522 whose outer diameter is smaller than the constraining section 521. The escaping tube 54 is mounted on the front section 531 of the plunger 53. The escape tube 54 is connected to the pulling ring 41 and driven by it to perform a reciprocal motion. At least one steel ball 59 (two in this embodiment) is disposed in the plunger 53 corresponding to the constraining holes 55. When the steel balls 59 are in contact with the constraining section 521 of the control pin 52, they urge against the rim of the escape tube 54 through the constraining holes 55 of the plunger 53. The plunger 53 and the escape tube 54 thus constrain each other. When the escape tube 54 moves along with the pulling ring 41 toward the urging surface 34 of the accommodating space 32 in the urging ring 31, the escape tube 54 concurrently pulls the plunger 53 to move toward the urging surface 34 and compress the second spring 56. When the steel balls 59 and the escaping section 522 of the control pin 52 are in contact, the steel balls 59 urge against the rim of the escape tube 54 without through the constraining holes 55. In this case, the plunger 53 is driven by the resilient force of the second spring 56 to perform a firing action.

In this embodiment, the gun body 12 of the body 11 is disposed with a fixing base 17. A tube 18 penetrates inside the fixing base 17. One end of the tube 18 accommodates the impact set 51 and goes into the connecting hole 43 of the pulling ring 41. The tube 18 of the impact set 51 is formed with a sliding groove 181. A through hole 541 is formed on the escape tube 54. A long axial rail groove 533 is formed on the outer side of the plunger 53. A fixing element 45 is fixed on the pulling ring 41 by its one end. Its other end goes through the sliding groove 181 of the tube 18. Via the through hole 541 of the escape tube 54, the fixing element 45 is accommodated in the rail groove 533 of the plunger 53. Therefore, the escape tube 54 and the pulling ring 41 are connected to perform a reciprocal motion along the sliding groove 181 of the tube 18.

One end of the urging pin set 61 is opposite to the impact set 51. Its other end is opposite to the hammer set 71. When the plunger 53 of the impact set 51 performs a firing action, the urging pin set 61 is hit by the plunger 53 to push the hammer set 71, producing an ejecting effect. The urging pin set 61 consists of an urging pin tube 62 and an urging pin 63. The urging pin tube 62 has a central hole 621 along its axial direction. The urging pin 63 goes into the central hole 621. One end of the urging pin 63 has an urging pin head 64, and the other end has a stop ring 65. The urging pin head 64 is opposite to the hammer set 71, and the stop ring 65 is opposite to the impact set 51. An urging pin spring 66 is interposed between the stop ring 65 and the urging pin tube 62, urging the stop ring 65 to push the urging pin 63 toward the impact set 51. An adjusting bar 67 is disposed in the direction from the urging pin head 64 to the urging pin tube 62. The central hole 621 of the urging pin tube 62 is surrounded by several adjusting holes 68, the depths of which are all different. An adjusting tube 69 is mounted outside the urging pin head 64. By rotating the adjusting tube 69, the urging pin head 64 is brought opposite to one of the adjusting holes 68.

The hammer set 71 includes a hammer ring 72, a hammer rod 73, and a hammer head 74. The hammer rod 73 goes through the hammer ring 72. One end of the hammer rod 73 is formed with a hitting surface 75 opposite to the urging pin head 64. The other end of the hammer rod 73 is connected with the hammer head 74. A hammer set spring 76 is interposed between the hammer head 74 and the hammer ring 72. The hammer head 74 is further mounted with a hammer tube 77 made of a soft material.

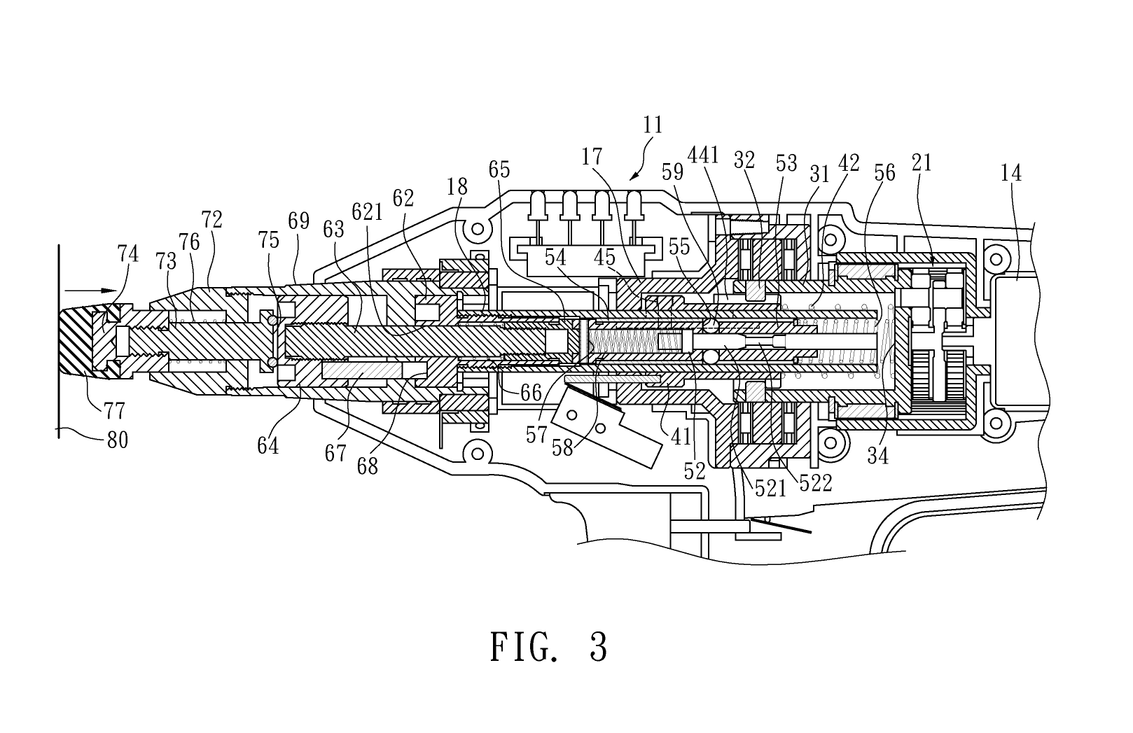

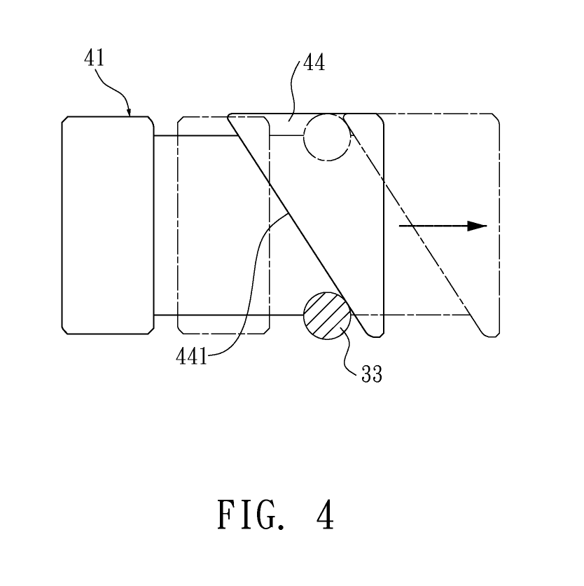

Please refer to FIG. 3. When the invention is in use, the user presses the hammer set 71 against his body 80. The hammer head 74 of the hammer set 71 depresses the hammer set spring 76 to move toward the urging pin set 61. Afterwards, the user presses the depressing part 161 to turn on the micro switch 162, electrically connecting the driving set 14 and the power supply 15. The driving set 14 thus starts to act. Through the gear set 21, the driving set 14 rotates the urging ring 31. In this case, the steel balls 59 are on the constraining section 521 of the control pin 52 and urge against the rim of the escape tube 54 through the constraining holes 55 of the plunger 59. The plunger 53 and the escape tube 54 thus constrain each other. When the urging ring 31 rotates, as shown in FIG. 4, the actuating parts 33 of the urging ring 31 push against the urging slant surfaces 441 of the pulling ring. The pulling ring 41 is forced to move toward the urging surface 34 of the urging ring 31. Since the escape tube 54 is connected with the pulling ring 41 via the fixing element 45, the escape tube 54 can move concurrently with the pulling ring 41. At the same time, the plunger 53 constrained with the escape tube 54 is pushed. The control pin 52 inside the plunger 53 moves toward the urging surface 34 of the urging ring 31. This compresses the first spring 42 and the second spring 56. The urging pin spring 66 of the urging pin set 61 urges the stop ring 65 to push the urging pin 63 toward the impact set 51.

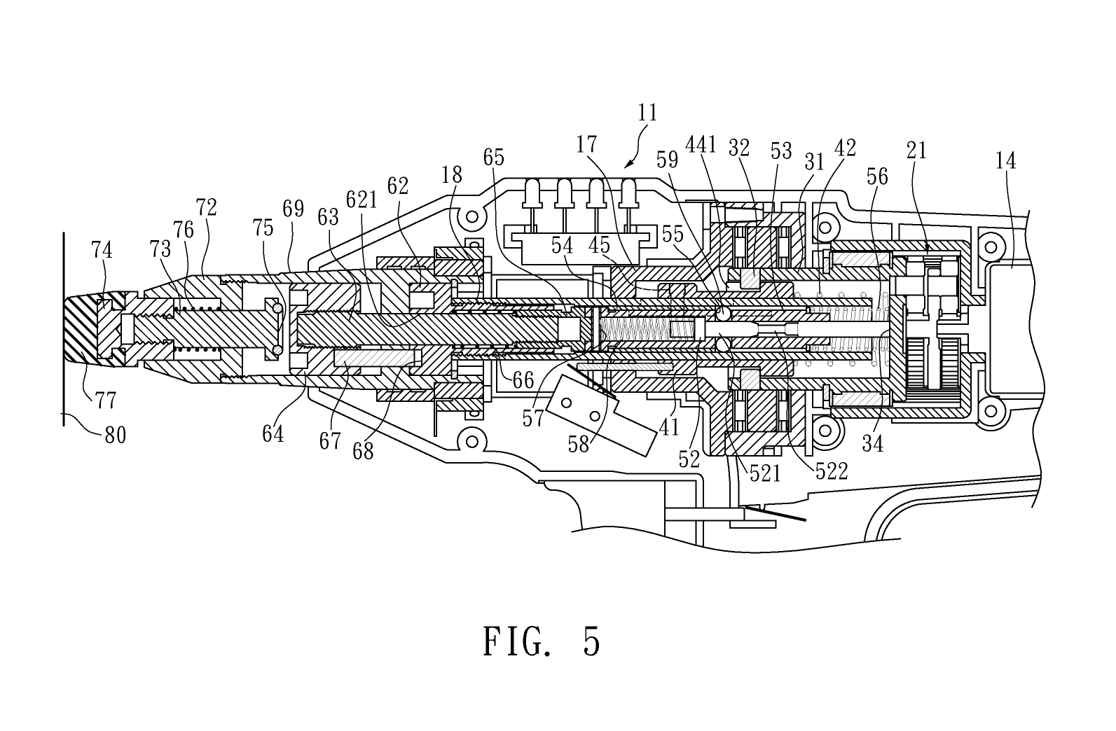

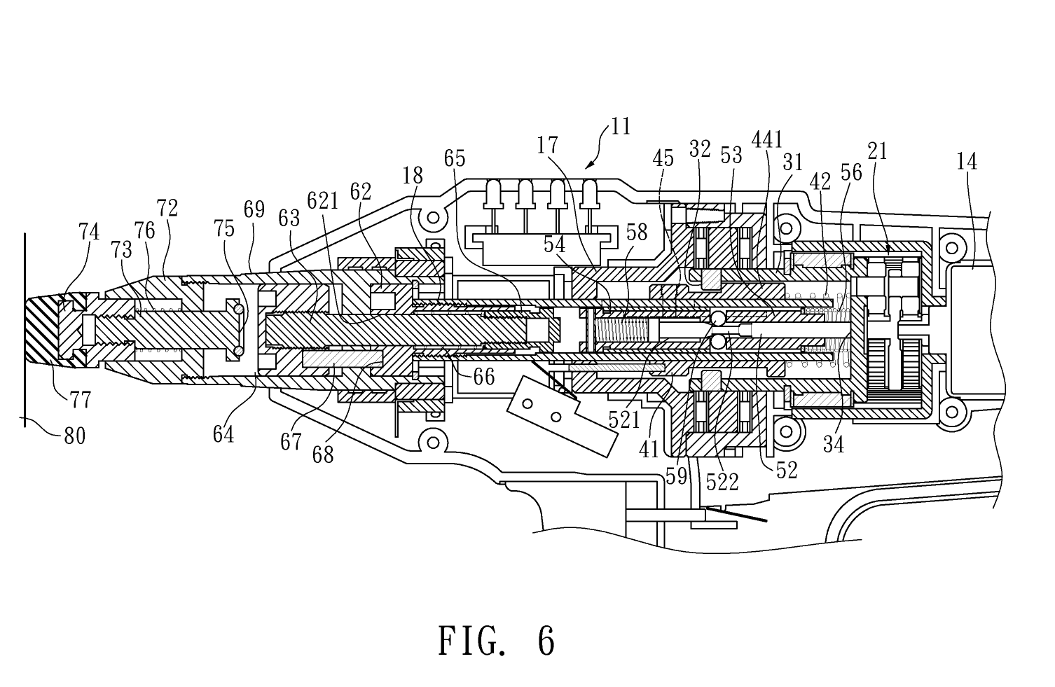

As shown in FIG. 5, when the control pin 52 continues to move toward the urging surface 34 of the urging ring 31 and urges against the urging surface 34, the control pin 52 is constrained by the urging surface 34 and cannot continue its motion. As shown in FIG. 6, the plunger is still driven by the escape tube 54 to push the steel balls 59 from the constraining section 521 of the control pin 52 to its escaping section 522. Since the outer diameter of the escaping section 522 of the control pin 52 is smaller than the constraining section 521, the constraining state between the escape tube 54 and the plunger 53 is released. In this embodiment, the steel balls 59 on the control pin 52 are squeezed by the plunger 52 into the constraining section 521. This releases the constraining state between the escape tube 54 and the plunger 53.

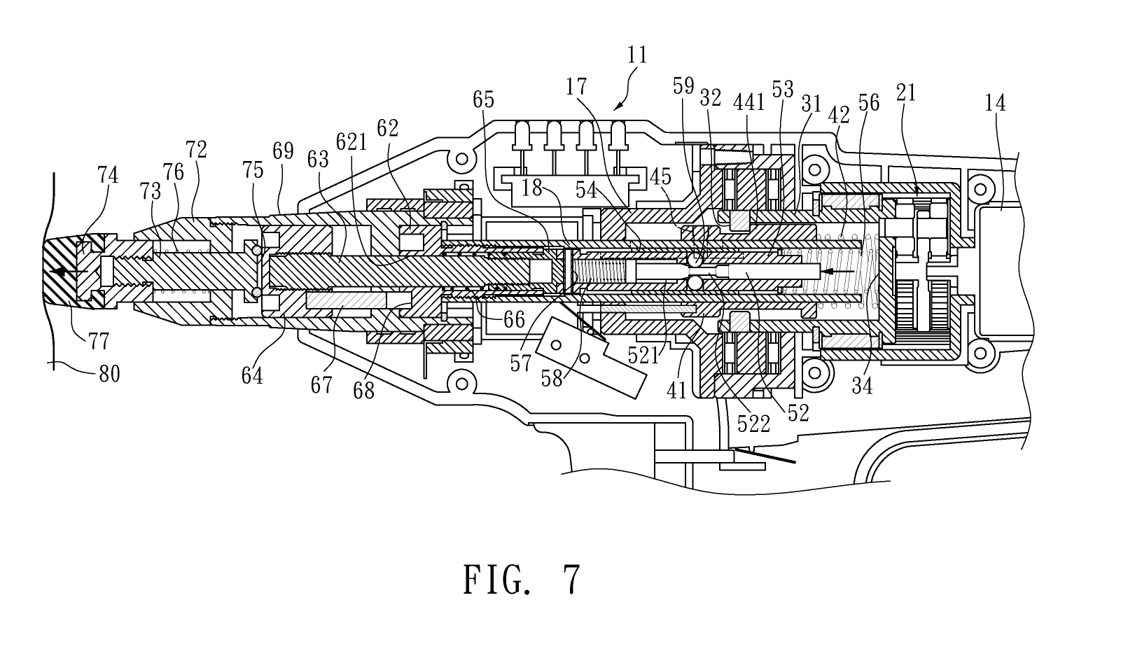

With reference to FIG. 7, when the escape tube 54 and the plunger 53 no longer constrain each other, the plunger 53 is driven by the resilient force of the second spring 56 to performing a firing action. It hits the stop ring 65 of the urging pin set 61, driving the urging pin head 64 to push the hammer head 74 of the hammer set 71 for generating an ejecting effect. This produces a massage effect on the human body. In this case, the escape tube 54 is still under the influence of the pulling ring 41 to move toward the urging surface 34 of the urging ring 31.

When the actuating parts 33 of the urging ring 31 leave the urging slant surfaces 441 of the pulling ring 41, the pulling ring 41 is driven by the resilient force of the first spring 42 to bring the escape tube 54 back to its original position. The steel balls 59 again urge against the rim of the escape tube 54 through the constraining hole 55 of the plunger 53. The escape tube 54 and the plunger 53 thus return to the constraining state (see FIG. 2). This completes an action of the disclosed power operated chiropractic adjuster.

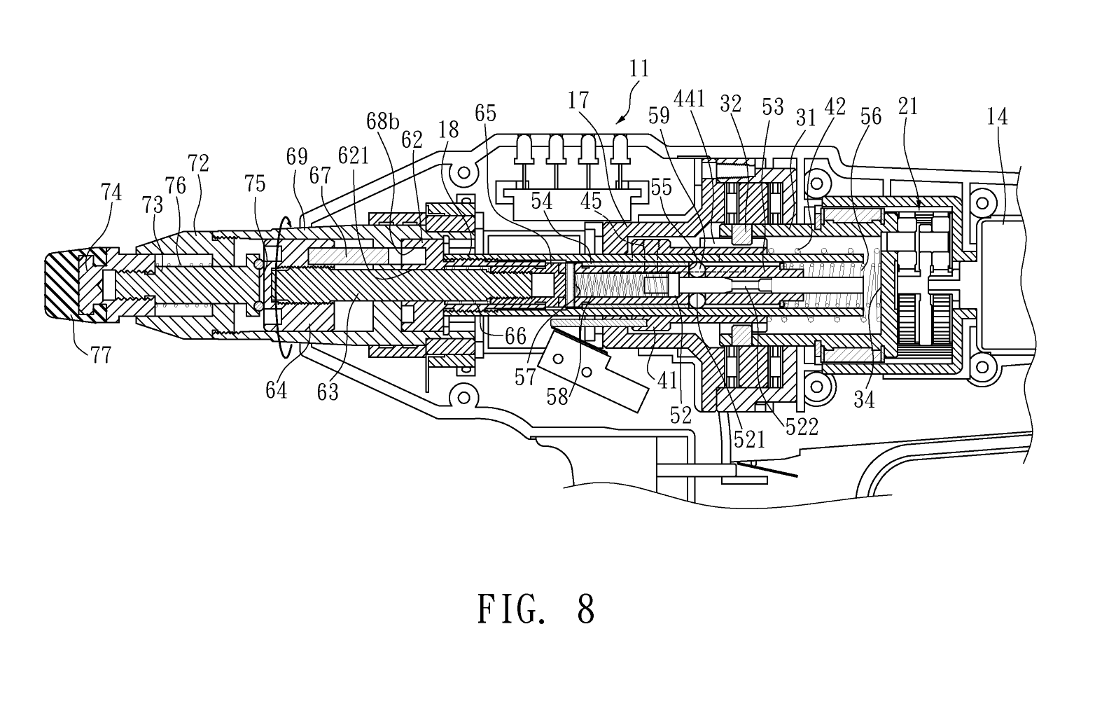

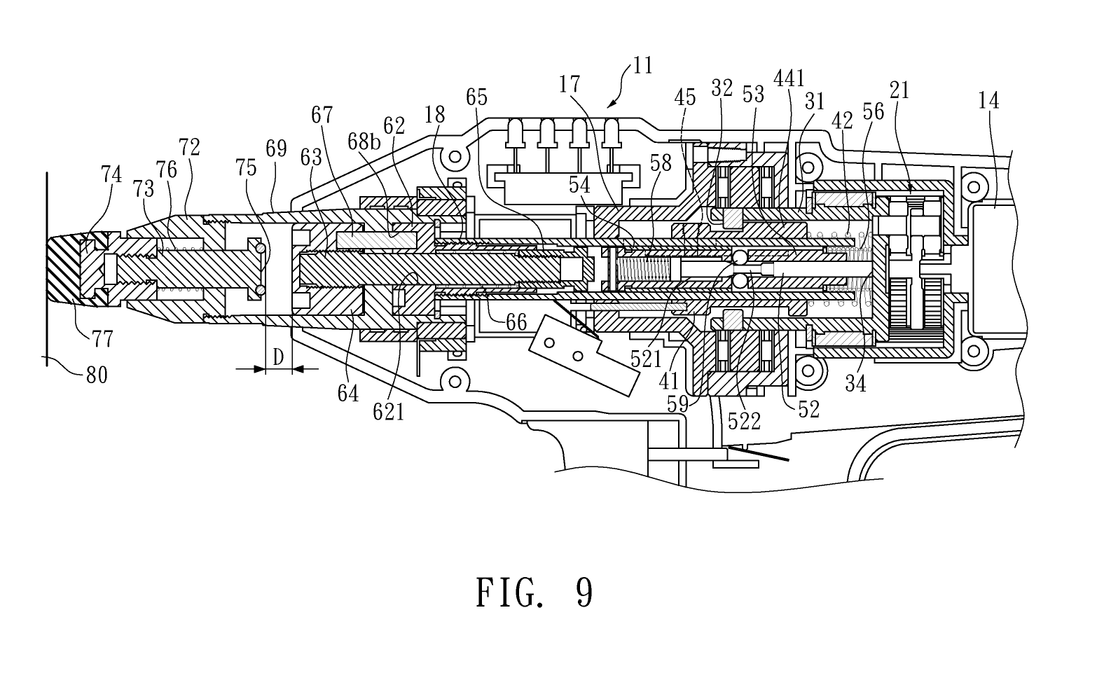

Please refer to FIG. 8. The user can rotate the adjusting tube 69 to let the adjusting bar 67 of the urging pin head 64 correspond to one of the adjusting holes 68b with a deeper depth. When the plunger 53 of the impact set 51 moves toward the urging surface 34 of the urging ring 31, as shown in FIG. 9, the urging pin 63 of the urging pin set 61 is driven by the urging pin spring 66 to move toward the impact set 51. The adjusting bar 67 of the urging pin head 64 is embedded into the adjusting hole 68b. As the adjusting hole 68b has a larger depth, the urging pin head 64 can move further toward the impact set 51. This increases the impact distance D between the urging pin head 64 and hitting surface 75 of the hammer set 71. Therefore, when the urging pin head 64 hits against the hammer set 71, a larger impact force is produced.

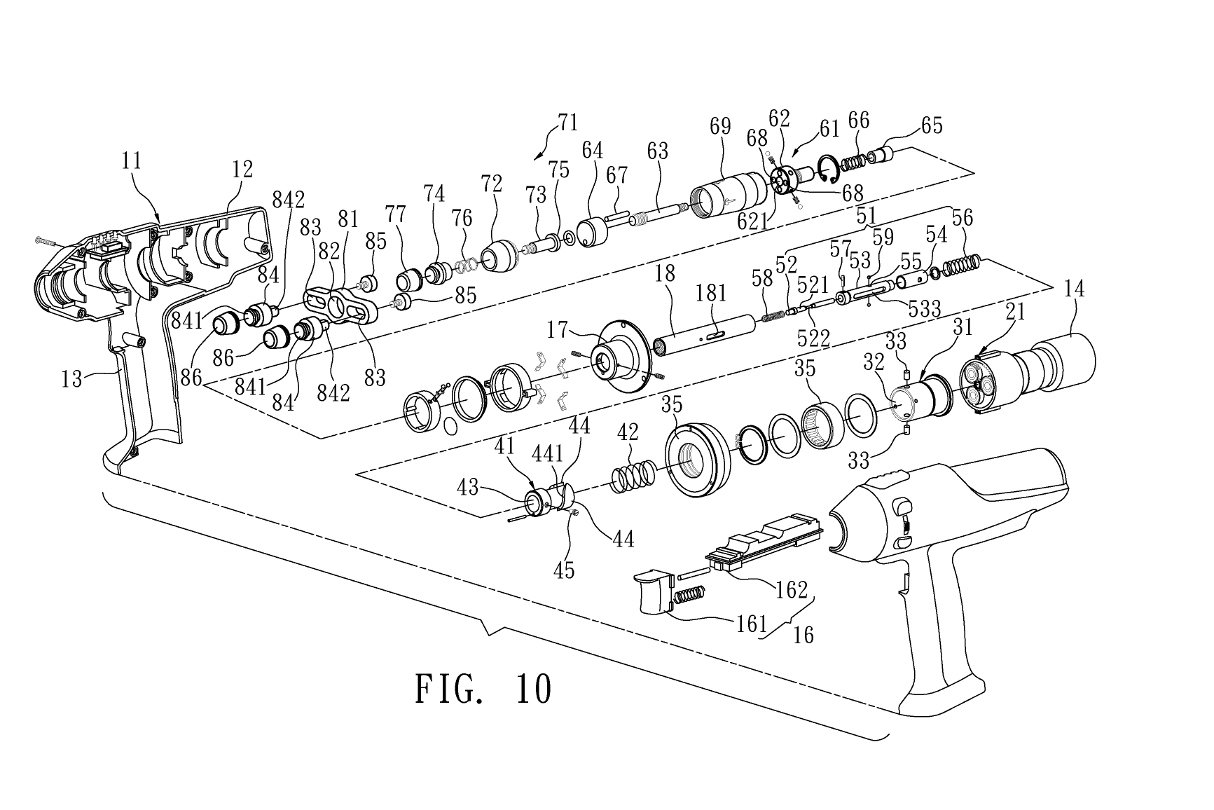

Please refer to FIG. 10 for a second embodiment of the invention. It differs from the above embodiment in the following aspects.

The hammer tube 77 of the hammer set 71 is further mounted with a mounting element 81. The mounting element 81 has a mounting hole 82 for the hammer tube 77 of the hammer head 74 to connect. Both sides of the mounting hole 82 are protruded outward to form a groove 83, respectively. Each of the grooves 83 is disposed with a side hammer 84. Each of the side hammers 84 has an impact end 841 and a connect end 842 opposite to the impact end 841. Each of the side hammers 84 goes into the corresponding groove 83 using its connect end 842 and move therein. When the connect end 842 of each of the side hammers 84 goes into the corresponding groove 83, it is locked using a fixing element 85 so that each side hammer 84 is positioned in the corresponding groove 83. Moreover, the impact end 841 of each side hammer 84 is mounted with a hammer tube 86 made of a soft material.

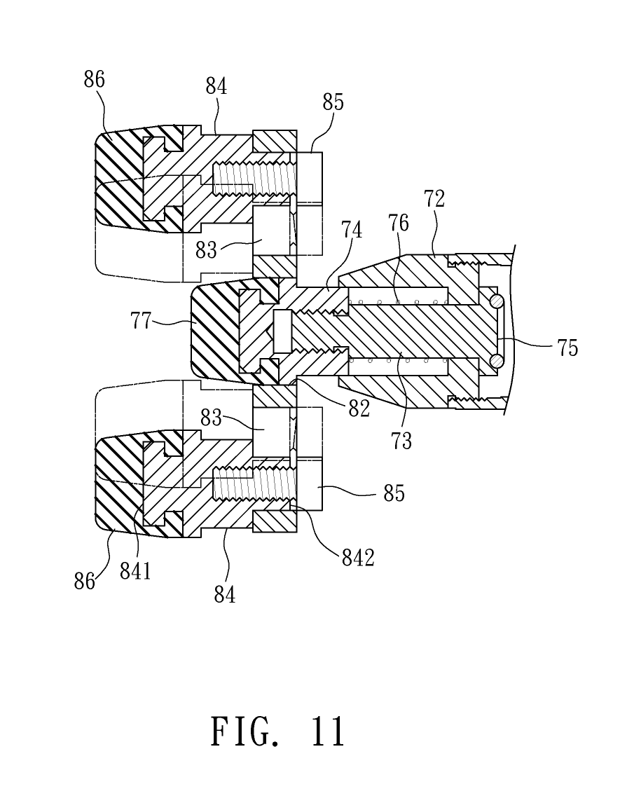

Please refer to FIG. 11. When the locking state of the fixing elements 85 on the side hammers 84 is released, the user is allowed to adjust the distance between the two side hammers 84. Through this mechanism, the user can readily and quickly adjust the distance between the two side hammers 84 according his build or special need.

In accord with the above-mentioned composition, the invention has the following advantages:

1. The impact set 51 determines the constraining state between the plunger 53 and the escape tube 54 through the control pin 52. The plunger 53 of the impact set 51 first performs a firing action to buffer the instantaneous impact of the chiropractic adjuster. The massaging force produced by the disclosed power operated chiropractic adjuster is smoother, such that no injury to the human spine will occur.

2. The disclosed power operated chiropractic adjuster adopts the design that there are multiple adjusting holes 68 of different depths on the urging pin tube 62 of the urging pin set 61. When the user rotates the adjusting tube 69 so that the adjusting bar 67 of the urging pin head 64 embeds into one of the adjusting holes 68, the impact distance between the urging pin head 64 and the hitting surface 75 of the hammer set 74 can be changed according to the depth of the adjusting hole 68. This achieves the function of modifying the strength of massaging force.

3. The invention has an independent power supply 15 inside its body. The power supply 15 provides the electrical power required for the action of the driving set 14. Therefore, the disclosed power operated chiropractic adjuster can be used at any place without environmental limitations. Moreover, the design of a built-in independent power supply 15 prevents the invention from interference of a power cord. This is particular convenient for operations.

4. The invention has further a mounting element 81 with two side hammers 84. The two side hammers 84 move along the corresponding grooves 83. Using this mechanism, the user can readily and quickly adjust the distance between the two side hammers 84 according to his build or special need, making the invention suitable for people of all builds.

Although the invention has been described with reference to specific embodiments, this description is not meant to be construed in a limiting sense. Various modifications of the disclosed embodiments, as well as alternative embodiments, will be apparent to people skilled in the art. Therefore, it is contemplated that the appended claims will cover all modifications that fall within the true scope of the invention.

What is claimed is:

1. A power operated chiropractic adjuster, comprising:
a body, whose inside has a driving set and a power supply that are electrically connected with each other;
an urging ring, whose one end is connected to the driving set for the driving set to rotate the urging ring and whose other end has an accommodating space, the accommodating space having two oppositely disposed actuating parts and an urging surface;
a pulling ring, which is disposed in the accommodating space of the urging ring with a first spring interposed between the pulling ring and the urging surface of the accommodating space, has a connecting hole formed along its axial direction, and has two actuating blocks disposed around the rim thereof; wherein each of the actuating blocks has an urging slant surface so that the two actuating parts on the urging ring engage with the two urging slant surfaces of the two actuating blocks, respectively, the actuating parts on the urging ring push the urging slant surfaces of the pulling ring as the urging ring is rotated by the driving set, forcing the pulling ring to move along its axial direction toward the urging surface of the urging ring to gradually compress the first spring, and the pulling is driven by the resilient force of the first spring back to its original position when the actuating parts of the urging ring leave the urging slant surfaces of the pulling ring;
an impact set disposed in the connecting hole of the pulling ring, which includes a control pin, a hollow plunger, and an escape tube mounted in sequence; wherein the plunger has a front section and a rear section and at least one constraining hole, a second spring is interposed between the rear section of the plunger and the urging surface of the urging ring, the control pin is disposed inside the plunger and has a constraining section and an escaping section, the escape tube is mounted on the front section of the plunger and connected with the pulling ring to perform a reciprocal motion with it, at least one steel ball is disposed inside the plunger to correspond to the constraining holes, the steel balls urge against the rim of the escape tube through the constraining holes of the plunger when they are in contact with the constraining section of the control pin, making the plunger and the escape tube constrain each other, the escape tube concurrently pulls the plunger to move toward the urging surface of the accommodating space and compress the second spring as the escape tube moves along with the pulling ring toward the urging surface, the steel balls urge against the rim of the escape tube without through the constraining holes when the they are in contact with the escaping section of the control pin, so that the plunger performs a firing action under the resilient force of the second spring;
an urging pin set, whose one end is disposed opposite to the impact set and whose other end is disposed opposite to a hammer set, wherein the urging pin set is hit by the plunger to push the hammer set, producing an ejecting effect, when the plunger of the impact set performs the firing action.

2. The power operated chiropractic adjuster of claim 1, wherein a gear set consisting of a plurality of decelerating gears is interposed between the driving set and the urging ring so that the driving set first drives the gear set and then the gear set rotates the urging ring.

3. The power operated chiropractic adjuster of claim 1, wherein a pin goes through the front section of the plunger and a third spring is interposed between the pin and the control pin.

4. The power operated chiropractic adjuster of claim 1, wherein the body has a fixing base inside, the fixing base has a tube through it, one end of the tube accommodates the impact set and goes into the connecting hole of the pulling ring, a sliding groove is formed on the tube of the impact set, a through hole is formed on the escape tube, a long axial rail groove is formed on the outer side of the plunger, a fixing element is fixed on the pulling ring by its one end, and its other end goes through the sliding groove of the tube and is accommodated in the rail groove of the plunger via the through hole of the escape tube, so that the escape tube and the pulling ring are connected for performing a reciprocal motion along the sliding groove of the tube.

5. The power operated chiropractic adjuster of claim 1, wherein the urging pin set includes an urging pin tube having a central hole along its axial direction and an urging pin disposed in the central hole, an urging pin head is disposed on one end of the urging pin and a stop ring on the other end, the urging pin head is opposite to the hammer set and the stop ring to the impact set, and an urging pin spring is interposed between the stop ring and the urging pin tube for urging the stop ring to push the urging pin toward the impact set.

6. The power operated chiropractic adjuster of claim 5, wherein an adjusting bar is disposed in the direction from the urging pin head to the urging pin tube, the central hole of the urging pin tube is surrounded by a plurality of adjusting holes of different depths, an adjusting tube is mounted outside the urging pin head, and the adjusting tube rotates so that the urging pin head is brought to correspond to one of the adjusting holes.

7. The power operated chiropractic adjuster of claim 1, wherein the body further has an ON/OFF switch electrically connected with the driving set and the power supply to control the electrical conduction between them and the driving set rotates the urging ring when the driving set and the power supply are in electrical communications.

8. The power operated chiropractic adjuster of claim 7, wherein the ON/OFF switch consists of a depressing part and a micro switch controlled by the depressing part, the depressing part exposes from the body, and the micro switch is disposed inside the body.

9. The power operated chiropractic adjuster of claim 1, wherein the hammer set includes a hammer ring, a hammer rod, and a hammer head with the hammer rod going through the hammer ring, one end of the hammer rod being formed with a hitting surface opposite to the urging pin set and the other end thereof being connected with the hammer head, and a hammer set spring is interposed between the hammer head and the hammer ring.

10. The power operated chiropractic adjuster of claim 9, wherein the hammer head is further mounted with a hammer tube made of a soft material.

11. The power operated chiropractic adjuster of claim 9, wherein the hammer tube of the hammer head is further mounted with a mounting element, the mounting element has a mounting hole for the connection of the hammer tube, both sides of the mounting hole are respectively protruded outward with a groove, each of which has a side hammer with an impact end and a connect end opposite to the impact end, each of the side hammers goes into the corresponding groove by its connect end, and a fixing element is used to lock the connect end of each of the side hammers, thereby positioning the side hammers.
